(19) United States
(12) Patent Application Publication
(10) Pub. No.: US 2021/0059918 A1
(43) Pub. Date: Mar. 4, 2021

(54) Oral Care Compositions Having Increased Foam Production and Methods for the Same

(71) Applicant: Colgate-Palmolive Company, New York, NY (US)

(72) Inventors: Aradhana DAS, Raritan, NJ (US); Hongwei SHEN, Holmdel, NJ (US); Eric SIMON, Princeton, NJ (US); Yun XU, Langhorne, PA (US)

(73) Assignee: Colgate-Palmolive Company, New York, NY (US)

(21) Appl. No.: 17/003,446

(22) Filed: Aug. 26, 2020

Related U.S. Application Data
(60) Provisional application No. 62/892,243, filed on Aug. 27, 2019.

(51) Int. Cl. A61K 8/46; A61K 8/19; A61K 8/60; A61Q 11/00; A61K 8/44

(52) U.S. Cl. CPC A61K 8/466; A61K 8/19; A61K 8/604; A61Q 11/00; A61K 2800/30; A61K 2800/596; A61K 2800/92; A61K 2800/28; A61K 8/44

(57) ABSTRACT

This invention relates to oral care compositions with improved foaming properties which comprise a basic amino acid, an abrasive and a taurate surfactant, as well as to methods of using these compositions. Optionally, the oral care compositions further comprise an alkyl polyglucoside.

### BACKGROUND

Dentin or dentinal hypersensitivity is a common clinical condition associated with exposed dentin surfaces of the teeth. Dentin contains a large number of pores or dentin tubules that extend from outer surfaces of the teeth to the nerves within the teeth. As such, exposure of the dentin often leads to increased sensitivity of the teeth to external stimuli (e.g., temperature, pressure, etc.). In view of the foregoing, conventional oral care products or compositions thereof may often attempt to numb the nerve or incorporate filling or blocking agents to ameliorate the sensitivity of the teeth. For example, conventional oral care compositions, such as Colgate Sensitive Pro-Relief®, often include arginine and calcium carbonate as blocking agents to occlude the dentin and reduce sensitivity.

While conventional oral care compositions have proven to be effective in reducing sensitivity, the active ingredients (e.g., arginine, calcium carbonate, etc.) may often react with one or more foaming agents of the oral care compositions to thereby reduce the ability of the oral care compositions to generate foam. For example, conventional oral care compositions primarily utilize anionic surfactants, such as sodium lauryl sulfate (SLS), as the primary foaming agent. The anionic surfactants, however, react with the cationically charged arginine and the calcium cations provided by the calcium carbonate to produce insoluble salts, thereby reducing the availability of the foaming agent and the foaming capacity thereof. Further, consumer studies and surveys have shown that there is a desire to use oral care compositions that do not contain any sodium lauryl sulfate, as some consumers experience relatively greater sensitivity to this ingredient.

What is needed, then, are improved desensitizing oral care compositions and methods for the same.

### BRIEF SUMMARY

This summary is intended merely to introduce a simplified summary of some aspects of one or more embodiments of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The present disclosure provides an oral care composition, e.g., toothpaste or gel, comprising:

an orally acceptable vehicle;

a taurate surfactant represented by formula (1)

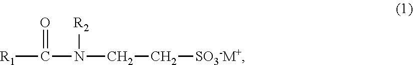

wherein R₁ is a saturated or unsaturated, straight or branched alkyl chain with 6 to 18 C atoms R₂ is H or methyl, and M⁺ is H, sodium, or potassium;

a basic amino acid, in free or salt form; and

an abrasive.

In some embodiments, the abrasive comprises a calcium-containing abrasive, e.g., calcium carbonate or dicalcium phosphate dihydrate. In some embodiments, the basic amino acid is arginine.

In some embodiments, the oral care composition further comprises a nonionic surfactant. In a preferred embodiment, the nonionic surfactant comprises a C6-C24 alkyl polyglucoside.

The present disclosure further provides a method comprising applying an effective amount of an oral care composition as disclosed herein to the oral cavity, e.g., by brushing, to a subject in need thereof, to (i) reduce or inhibit formation of dental caries, (ii) reduce, repair or inhibit pre-carious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the oral cavity, (vii) reduce levels of acid producing bacteria, (viii) reduce or inhibit microbial biofilm formation in the oral cavity, (ix) reduce or inhibit plaque formation in the oral cavity, (x) promote systemic health, or (xi) clean teeth and oral cavity. In a preferred embodiment, the present disclosure provides a method for treating dentinal hypersensitivity in a human or animal subject with an oral care composition as disclosed herein.

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### DETAILED DESCRIPTION

The following description of various typical aspect(s) is merely exemplary in nature and is in no way intended to limit the disclosure, its application, or uses.

As used throughout this disclosure, ranges are used as shorthand for describing each and every value that is within the range. It should be appreciated and understood that the description in a range format is merely for convenience and brevity, and should not be construed as an inflexible limitation on the scope of any embodiments disclosed herein. Accordingly, the disclosed range should be construed to have specifically disclosed all the possible subranges as well as individual numerical values within that range. As such, any value within the range may be selected as the terminus of the range. For example, description of a range such as from 1 to 5 should be considered to have specifically disclosed subranges such as from 1.5 to 3, from 1 to 4.5, from 2 to 5, from 3.1 to 5, etc., as well as individual numbers within that range, for example, 1, 2, 3, 3.2, 4, 5, etc. This applies regardless of the breadth of the range.

Unless otherwise specified, all percentages and amounts expressed herein and elsewhere in the specification should be understood to refer to percentages by weight. The amounts given are based on the active weight of the material.

Additionally, all numerical values are “about” or “approximately” the indicated value, and take into account experimental error and variations that would be expected by a person having ordinary skill in the art. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

As used herein, “free” or “substantially free” of a material may refer to a composition, component, or phase where the material is present in an amount of less than 10.0 weight %, less than 5.0 weight %, less than 3.0 weight %, less than 1.0 weight %, less than 0.1 weight %, less than 0.05 weight %, less than 0.01 weight %, less than 0.005 weight %, or less than 0.0001 weight % based on a total weight of the composition, component, or phase.

All references cited herein are hereby incorporated by reference in their entireties. In the event of a conflict in a definition in the present disclosure and that of a cited reference, the present disclosure controls.

The present inventors have surprisingly found that the combination of a taurate surfactant or a salt thereof and arginine is superior in terms of foam amount when compared to SLS and arginine combination. Without intending to be bound to theory, it is believed that as taurate is an anionic surfactant with large head group in comparison with SLS and arginine is amphoteric in nature at basic pH, there is less likely possibility of having an interaction between the two, therefore resulting in improved foaming. It has also been found that CaCl₂ (source of soluble calcium ions) and arginine solution when added to SLS solution yields a white precipitate of (Ca(H₂Arg)₂)(LS)₄ products, whereas no precipitate is formed when CaCl₂ and arginine solution is reacted with a taurate surfactant. Thus, taurate surfactant is a better alternative to sodium lauryl sulfate in the presence of arginine and calcium carbonate in order to increase foam production

The present inventors have also surprisingly found that oral care compositions including the combination of a taurate surfactant or a salt thereof and an alkyl polyglucoside surfactant exhibit synergistic or more than additive foaming as compared to each of the surfactants alone. It has also been found that relatively greater amounts of the nonionic polyglucoside surfactant to the anionic taurate surfactant or the salt thereof produces more foam as compared to toothpaste compositions including relatively greater amounts of the anionic alkyl methyl taurate salt. This is surprising, because an alkyl polyglucoside surfactant alone exhibits poor foaming activity. Accordingly, it has been surprisingly found that increased amounts or ratios of the nonionic surfactant relative to the anionic taurate surfactant or the salt thereof provides improved foaming. Without being bound by theory, it is believed that the nonionic structure of the glucoside surfactant and the negatively charged structure of the taurate surfactant or the salt thereof may contribute to the synergistic results. It is believed that when both are used together, they form mixed micelles, thus resulting in some synergistic effect.

The present invention provides, in an aspect, an oral care composition (Compositions 1.0), e.g., toothpaste or gel, comprising:

an orally acceptable vehicle;

a taurate surfactant represented by formula (1)

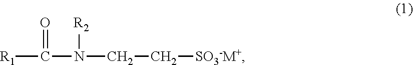

wherein R₁ is a saturated or unsaturated, straight or branched alkyl chain with 6 to 18 C atoms R₂ is H or methyl, and M⁺ is H, sodium, or potassium;

a basic amino acid, in free or salt form; and

an abrasive, e.g., calcium carbonate.

For example, the invention includes:
1.1. Composition 1.0, wherein the R₁ is a saturated or unsaturated, straight or branched alkyl chain with 8 to 14 C atoms.
1.2. Composition 1.0 or 11, wherein the taurate surfactant comprises one or more of potassium cocoyl taurate, potassium methyl cocoyl taurate, sodium caproyl methyl taurate, sodium cocoyl taurate, sodium lauroyl taurate, sodium methyl cocoyl taurate, sodium methyl lauroyl taurate, sodium methyl myristoyl taurate, sodium methyl oleoyl taurate, sodium methyl palmitoyl taurate, sodium methyl stearoyl taurate, or combinations and mixtures thereof.
1.3. Any of the preceding compositions, wherein the taurate surfactant comprises sodium lauroyl methyl taurate (or sodium methyl lauroyl taurate), sodium methyl cocoyl taurate (sodium cocoyl methyl taurate), or combinations thereof.
1.4. Any of the preceding compositions, wherein the taurate surfactant is sodium lauroyl methyl taurate.
1.5. Any of the preceding compositions, wherein the taurate surfactant is sodium methyl cocoyl taurate.
1.6. Any of the preceding compositions, wherein the taurate surfactant is present in an amount of from 0.4% to 3%, e.g., from 0.4% to 2.5%, from 0.4% to 2%, from 0.4% to 1.5%, from 0.5% to 3%, from 0.8% to 3%, from 1% to 3%, from 1.2% to 2.7%, from 1.5% to 3%, from 2% to 3%, from 1% to 2.8%, from 1% to 2.7%, from 1% to 2.5%, from 1.5% to 2.8%, from 1.5% to 2.5%, from 1.8% to 3%, from 1.8% to 2.8%, from 1.8% to 2.'7%, from 1.8% to 2.5%, from 2% to 2.8%, from 2% to 2.'7%, or from 2% to 2.5% by weight of the composition.
1.7. Any of the preceding compositions, wherein the composition further comprises a nonionic surfactant.
1.8. Composition 1.7, wherein the nonionic surfactant comprises a C6-C24 alkyl polyglucoside, e.g., a C6-C18 alkyl polyglucoside, optionally wherein the nonionic surfactant is a C6-C24 alkyl polyglucoside, e.g., a C6-C18 alkyl polyglucoside.
1.9. Composition 1.8, wherein the C6-C24 alkyl polyglucoside comprises a short chain alkyl polyglucoside, optionally wherein the short chain alkyl polyglucoside comprises a C6-C10 alkyl polyglucoside, and further optionally wherein the short chain alkyl polyglucoside comprises a C8-C10 alkyl polyglucoside.
1.10. Composition 1.8, wherein the C6-C24 alkyl polyglucoside comprises a long chain alkyl polyglucoside, optionally wherein the long chain alkyl polyglucoside comprises a C11-C24 alkyl polyglucoside.
1.11. Any of compositions 1.7 to 1.0, wherein the nonionic surfactant is present in an amount of 0.7% to 2.5%, 0.8% to 2.5%, 1% to 2.5%, 1.3% to 2.5%, 1.5% to 2.5%, 0.7% to 2%, 0.8% to 2%, 1% to 2%, 1.3% to 2%, 1.5% to 2%, 0.8% to 1.7%, 0.8% to 1.5%, 1% to 1.5%. 1.3% to 1.8%, 1.3% to 1.7%, or about 1.5% by weight of the composition.
1.12. Any of compositions 1.7 to 1.1, wherein a weight ratio of the nonionic surfactant to the taurate surfactant is from 0.25:1 to 9:1, e.g., from 1:1 to 5:1, from 1:1 to 2.5:1, from 1.3 to 2.5:1, from 1.3:1 to 2.2:1, from 1.3:1 to 2:1, from 1.4:1 to 1.8:1, from about 1.8:1 to about 2.2:1, from 1.4:1 to 2.1:1, from 1.4:1 to 1.6:1. or from 1.5:1 to 2:1. 1.13. Any of compositions 1.7 to 1.12, wherein the taurate surfactant is present in an amount of from 0.4% to 1.5% and the nonionic surfactant is present in an amount of 0.7% to 2% by weight of composition.
1.14. Any of compositions 1.7 to 1.12, wherein the taurate surfactant is present in an amount of from 0.5% to 1% and the nonionic surfactant is present in an amount of 1% to 1.5% by weight of composition.
1.15. Any of compositions 1.7 to 1.12, wherein the taurate surfactant is present in an amount of from 0.8% to 1.2% and the nonionic surfactant is present in an amount of 1.3% to 1.8% by weight of composition.
1.16. Any of compositions 1.7 to 1.12, wherein the taurate surfactant is present in an amount of about 1% and the nonionic surfactant is present in an amount of about 1.5 by weight of composition.
1.17. Any of the preceding compositions, wherein the basic amino acid comprises one or more of arginine, lysine, citrulline, ornithine, creatine, histidine, diaminobutanoic acid, diaminoproprionic acid, salts thereof, or combinations thereof.
1.18. Any of the preceding compositions, wherein the basic amino acid has the L-configuration.
1.19. Any of the preceding compositions, wherein the basic amino acid is present in an amount of from 1% to 15%, e.g., from 1% to 10%, from 3% to 10%, from 4% to 8%, from 5% to 7%, from 4% to 12%, from 4% to 10%, from 6% to 10%, about 8%, or about 6% by weight of the composition, being calculated as free base form.
1.20. Any of the preceding compositions, wherein the basic amino acid comprises arginine.
1.21. Any of the preceding compositions, wherein the basic amino acid comprises L-arginine.
1.22. Any of the preceding compositions, wherein the basic amino acid comprises arginine bicarbonate, arginine phosphate, arginine sulfate, arginine hydrochloride or combinations thereof, optionally wherein the basic amino acid is arginine bicarbonate.
1.23. Any of the preceding compositions, wherein the abrasive comprises a calcium-containing abrasive, optionally wherein the calcium-containing abrasive is selected from calcium carbonate, calcium phosphate (e.g., dicalcium phosphate dihydrate), calcium sulfate, and combinations thereof.
1.24. Any of the preceding compositions, wherein the abrasive comprises calcium carbonate, optionally wherein the calcium carbonate comprises precipitated calcium carbonate.
1.25. Any of the preceding compositions, wherein the abrasive comprises calcium phosphate (e.g., dicalcium phosphate dihydrate).
1.26. Any of the preceding compositions, wherein the abrasive is present in an amount of from 15% to 70%, e.g., from 20% to 50%, from 25% to 45%, from 30% to 40%, or about 35% by weight of the composition.
1.27. Any of the preceding compositions, wherein the composition comprises an effective amount of a fluoride ion source, e.g., providing 500 to 3000 ppm fluoride.
1.28. Composition 1.27, wherein the fluoride ion source is a salt selected from stannous fluoride, sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and combinations thereof, optionally wherein the fluoride ion source is sodium fluoride or sodium monofluorophosphate.
1.29. Composition 1.27, wherein the fluoride ion source is sodium monofluorophosphate.
1.30. Any of the preceding compositions, wherein the composition comprises one or more soluble phosphate salts, e.g. selected from tetrasodium pyrophosphate (TSPP), sodium tripolyphosphate (STPP) and combinations thereof.
1.31. Any of the preceding compositions, wherein the composition further comprises a zinc ion source.
1.32. Composition 1.31, wherein the zinc ion source is selected from the group consisting of zinc oxide, zinc sulfate, zinc chloride, zinc citrate, zinc lactate, zinc gluconate, zinc malate, zinc tartrate, zinc carbonate, zinc phosphate and a combination thereof.
1.33. Combination 1.31 or 1.32, wherein the zinc ion source is selected from the group consisting of zinc oxide, zinc citrate, and a combination thereof.
1.34. Any of Compositions 1.31 to 1.33, wherein the zinc ion source is present an amount of from 0.01% to 5%, e.g., 0.1% to 4%, or 0.5% to 3%, by weight of the composition.
1.35. Any of the preceding compositions, wherein the composition is substantially free of sodium lauryl sulfate.
1.36. Any of the preceding compositions, wherein the composition is substantially free of alkyl sulfate salts.
1.37. Any of the preceding compositions, wherein the composition is a toothpaste or gel.
1.38. Any of the preceding compositions, wherein the composition is a toothpaste.
1.39. Any of the preceding composition, wherein the composition comprises sodium lauroyl methyl taurate and a C8-C10 alkyl polyglucoside.
1.40. The composition of 1.39, wherein the sodium lauroyl methyl taurate is in an amount from 0.25% -1.25% by wt. of the composition (e.g., 0.5% SLMT) (e.g, 1.0% SLMT) and the C8-C10 alkyl polyglucoside is in an amount from 0.75% -1.75% by wt. of the composition (e.g., 1% by wt APG 08/10) (e.g., 1.5% by wt APG 08/10).
1.41. The composition of 1.40, wherein the total amount of sodium lauroyl methyl taurate and the C8-C10 alkyl polyglucoside is from 1.0%-3.0% by wt. of the total composition.
1.42. The composition of 1.41, wherein the total amount of sodium lauroyl methyl taurate and the C8-C10 alkyl polyglucoside is from 1.25%-2.75% by wt. of the total composition.
1.43. The composition of 1.42, wherein the total amount of sodium lauroyl methyl taurate and the C8-C10 alkyl polyglucoside is about 1.5% or about 2.5% by wt. of the total composition.
1.44. Any of the preceding compositions comprising:
   a. An orally acceptable vehicle;
   b. sodium lauroyl methyl taurate in an amount from 0.25%-1.25% by wt. of the composition;
   c. C8-C10 alkyl polyglucoside in an amount from 0.75%-1.75% by wt. of the composition;
   d. arginine (e.g., arginine bicarbonate); and
   e. calcium carbonate (e.g., precipitated calcium carbonate)
1.45. Any of the preceding compositions comprising:
   a. An orally acceptable vehicle;
   b. sodium lauroyl methyl taurate in an amount of about 0.5% by wt. of the composition;
   c. C8-C10 alkyl polyglucoside in an amount from about 1.0% by wt. of the composition
   d. arginine (e.g., arginine bicarbonate); and
   e. calcium carbonate (e.g., precipitated calcium carbonate)
1.46. Any of the compositions 1.0-1.45 comprising:
   a. An orally acceptable vehicle;
   b. sodium lauroyl methyl taurate in an amount of about 1.0% by wt. of the composition;
   c. C8-C10 alkyl polyglucoside in an amount from about 1.5% by wt. of the composition
   d. arginine (e.g., arginine bicarbonate); and
   e. calcium carbonate (e.g., precipitated calcium carbonate)

### Compositions

Compositions disclosed herein may be or include an oral care product or an oral care composition thereof. For example, the compositions disclosed herein may be an oral care product including an oral care composition or the oral care composition thereof. In some embodiments, the compositions disclosed herein may be or include oral care compositions including an orally acceptable vehicle or carrier, one or more basic amino acids (e.g., arginine), and one or more abrasives (e.g., calcium carbonate), and one or more surfactants or foaming agents capable of or configured to provide relatively greater foam production as compared to conventional oral care compositions. For example, compositions disclosed herein may be or include oral care compositions, such as toothpaste compositions for sensitive teeth, that include an orally acceptable vehicle or carrier and one or more surfactants capable of or configured to provide relatively greater foam production as compared to conventional oral care compositions, which utilize conventional anionically charged surfactants, such as sodium lauryl sulfate (SLS). In some embodiments, the one or more surfactants may include an anionic surfactant, wherein the anionic surfactant may be or include a taurate surfactant or a salt thereof. In another embodiment, the one or more surfactants may include an anionic surfactant and a nonionic surfactant, wherein the anionic surfactant may be or include a taurate surfactant or a salt thereof, and wherein the nonionic surfactant may be or include a C6-C24 (e.g., C6-C18) alkyl polyglucoside. In some embodiments, a weight ratio of the nonionic surfactant to the anionic surfactant may be from about 0.25:1 to about 9:1.

Illustrative oral care products or compositions of the present disclosure may be or include, but are not limited to, a toothpaste (dentifrice), a prophylactic paste, a tooth powder, or a tooth gel (e.g., a whitening gel). In an exemplary embodiment, the oral care composition disclosed herein may be a toothpaste or gel. For example, the oral care composition disclosed herein may be a toothpaste for treating teeth sensitivity or dentinal hypersensitivity.

The oral care product or the oral care composition thereof may be a single phase oral care product or a single phase oral care composition. For example, all the components of the oral care product or the oral care composition thereof may be maintained together with one another in a single phase and/or vessel. For example, all the components of the oral care product or the oral care composition thereof may be maintained in a single phase, such as a single homogenous phase. In another embodiment, the oral care product or the oral care composition thereof may be a multi-phase oral care product or a multi-phase oral care composition.

Water may also be present in the oral compositions of the invention. Water employed in the preparation of commercial oral compositions should be deionized and free of organic impurities. Water commonly makes up the balance of the compositions and includes about 10% to about 90%, about 20% to about 60% or about 10% to about 30% by weight of the oral compositions. This amount of water includes the free water which is added plus that amount which is introduced with other materials such as with sorbitol or any components of the invention.

### Basic Amino Acid

The oral care composition of the invention may include one or more basic amino acids. The one or more amino acids of the oral care composition may be in free or salt form. Illustrative basic amino acids that may be utilized in the oral care composition may include, but are not limited to, arginine, lysine, citrulline, ornithine, creatine, histidine, diaminobutanoic acid, diaminoproprionic acid, salts thereof and/or combinations thereof. The basic amino acids of the oral care composition may generally be present in the L-form or L-configuration. The basic amino acids may be provided as a salt of a di- or tri-peptide including the amino acid. In some embodiments, at least a portion of the basic amino acid present in the oral care composition is in the salt form. In some embodiments, the oral care composition includes at least arginine (e.g., L-arginine) or a source of arginine. Arginine may be provided as free arginine or a salt thereof. For example, Arginine may be provided as arginine phosphate, arginine hydrochloride, arginine sulfate, arginine bicarbonate, and mixtures or combinations thereof. The one or more basic amino acids may be provided as a solution or a solid. For example, the one or more basic amino acids may be provided as an aqueous solution. In a preferred embodiment, the one or more amino acids include or are provided by an arginine bicarbonate solution. For example, the amino acid may be provided by an about 40% solution of the one or more basic amino acids, such as arginine bicarbonate or alternatively called as arginine carbamate.

The amount or concentration of the one or more basic amino acids present in the oral care composition may vary widely. In some embodiments, the amount or concentration of the one or more amino acids may be from greater than 0.1 weight % to about 20 weight %, based on the total weight of the oral care composition, being calculated as free base form. In some embodiments, the basic amino acid is present in an amount of from 1% to 15%, e.g., from 1% to 10%, from 3% to 10%, from 4% to 8%, from 5% to 7%, from 4% to 12%, from 4% to 10%, from 6% to 10%, about 8%, or about 6% by weight of the composition, being calculated as free base form.

### Abrasive or Abrasive System

The oral care compositions may include one or more abrasives or an abrasive system including one or more abrasives. As used herein, the term “abrasive” may also refer to materials commonly referred to as “polishing agents”. Any orally acceptable abrasive may be used, but preferably, type, fineness (particle size), and amount of the abrasive may be selected such that the tooth enamel is not excessively abraded in normal use of the oral care composition.

The one or more abrasives may have a particle size or D50 of less than or equal to about 10 μm, less than or equal to about 8 μm, less than or equal to about 5 μm, or less than or equal to about 3 μm. The one or more abrasives may have a particle size or D50 of greater than or equal to about 0.01 μm, greater than or equal to about 0.05 μm, greater than or equal to about 0.1 μm, greater than or equal to about 0.5 μm, or greater than or equal to about 1 μm. Illustrative abrasives may include, but are not limited to, metaphosphate compounds, phosphate salts (e.g., insoluble phosphate salts), such as sodium metaphosphate, potassium metaphosphate, calcium pyrophosphate, magnesium orthophosphate, trimagnesium orthophosphate, tricalcium phosphate, dicalcium phosphate dihydrate, anhydrous dicalcium phosphate, calcium carbonate (e.g., precipitated calcium carbonate and/or natural calcium carbonate), magnesium carbonate, hydrated alumina, silica, zirconium silicate, aluminum silicate including calcined aluminum silicate, polymethyl methacrylate, or mixtures and combinations thereof.

In some embodiments, the oral care composition of the invention comprises a calcium-containing abrasive (e.g., calcium carbonate). In some embodiments, the calcium-containing abrasive is selected from calcium carbonate, calcium phosphate (e.g., dicalcium phosphate dihydrate), calcium sulfate, and combinations thereof. In a preferred embodiment, the oral care composition comprises calcium carbonate as an abrasive. In one embodiment, the oral care composition comprises precipitated calcium carbonate. Precipitated calcium carbonate may be preferred over natural calcium carbonate. While not intending to be bound by theory, it is believed that natural calcium carbonate has relatively greater crystallinity or a relatively more crystalline structure as compared to precipitated calcium carbonate, thereby making the calcium carbonate very hard. Conversely, precipitated calcium carbonate is relatively more amorphous and more friable or easily crumbled. As such, the precipitated calcium carbonate has a relatively lower abrasivity as compared to the natural calcium carbonate, while maintaining adequate cleaning power.

The one or more of the abrasives in the abrasive system may have a pellicle cleaning ratio (PCR) greater than or equal to 80, greater than or equal to 82, greater than or equal to 84, greater than or equal to 86, greater than or equal to 88, greater than or equal to 90, greater than or equal to 92, greater than or equal to 94, greater than or equal to 96, greater than or equal to 98, greater than or equal to 100, greater than or equal to 102, greater than or equal to 104, greater than or equal to 106, greater than or equal to 108, greater than or equal to 110, greater than or equal to 112, or greater.

The amount or concentration of the one or more abrasives present in the oral care composition may vary widely. In some embodiments, the amount or concentration of the abrasives may be from greater than 0 weight % to about 60 weight %, based on a total weight of the oral care composition. For example, the amount of the abrasives present in the oral care composition may be from greater than 0 weight %, about 2 weight %, about 4 weight %, about 6 weight %, about 8 weight %, about 10 weight %, about 12 weight %, about 14 weight %, about 16 weight %, about 18 weight %, or about 19 weight % to about 21 weight %, about 22 weight %, about 24 weight %, about 26 weight %, about 28 weight %, about 30 weight %, about 32 weight %, about 34 weight %, about 36 weight %, about 38 weight %, or about 40 weight %. In another example, the amount of the abrasives present in the oral care composition may be from greater than 0 weight % to about 40 weight %, about 2 weight % to about 38 weight %, about 4 weight % to about 36 weight %, about 6 weight % to about 34 weight %, about 8 weight % to about 32 weight %, about 10 weight % to about 30 weight %, about 12 weight % to about 28 weight %, about 14 weight % to about 26 weight %, about 16 weight % to about 24 weight %, about 18 weight % to about 22 weight %, or about 19 weight % to about 21 weight %. In a preferred embodiment, the amount of the abrasives present in the oral care composition may be from about 15 weight % to about 70 weight %, e.g., from about 20 weight % to about 50weight %, from about 25 weight % to about 45 weight %, preferably about 30 weight % to about 40 weight %, or more preferably about 35 weight %, based on a total weight of the oral care composition.

### Surfactant(s) or Foaming System

The oral care composition may include one or more surfactants or foaming agents. The one or more surfactants or foaming agents may be capable of or configured to provide relatively greater foam production as compared to conventional oral care compositions. The one or more surfactants or foaming agents may be or include, but are not limited to, one or more amino acid-based surfactants, one or more anionic surfactants, one or more cationic surfactants, one or more zwitterionic surfactants, one or more nonionic surfactants, or combinations and mixtures thereof. The amino acid surfactants may be or include, but are not limited to, taurate surfactants, alanine surfactants, aspartate surfactants, or any mixture or combination thereof. Illustrative taurate surfactants may be represented by formula (1):

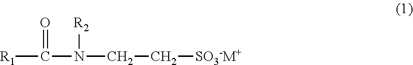

where R₁may be a saturated or unsaturated, straight or branched alkyl chain with 6 to 18 C atoms, and preferably 8 to 14 C atoms, R₂ may be H or methyl, and M⁺ may be H, sodium, or potassium. Illustrative taurate surfactants may be or include, but are not limited to, potassium cocoyl taurate, potassium methyl cocoyl taurate, sodium caproyl methyl taurate, sodium cocoyl taurate, sodium lauroyl taurate, sodium methyl cocoyl taurate, sodium methyl lauroyl taurate, sodium methyl myristoyl taurate, sodium methyl oleoyl taurate, sodium methyl palmitoyl taurate, sodium methyl stearoyl taurate, or combinations and mixtures thereof. In a preferred embodiment, the taurate surfactants may be or include sodium lauroyl methyl taurate (or sodium methyl lauroyl taurate), sodium methyl cocoyl taurate (or sodium cocoyl methyl taurate), or combinations thereof.

The anionic surfactants may be or include, but are not limited to, one or more C6-C18 fatty acid glutamate salts, water-soluble salts of C8-20 alkyl sulfates, sulfonated monoglycerides of C8-20 fatty acids, sarcosinates, taurates, sodium lauryl sulfate, sodium cocoyl monoglyceride sulfonate, sodium lauryl sarcosinate, sodium lauryl isoethionate, sodium laureth carboxylate, sodium lauroyl methyl taurate, sodium dodecyl benzenesulfonate, water-soluble salts of higher fatty acid monoglyceride monosulfates, such as the sodium salt of the monosulfated monoglyceride of hydrogenated coconut oil fatty acids such as a sodium N-methyl-N-alkyl taurate, sodium N-methyl-N-cocoyl taurate or sodium methyl cocoyl taurate, sodium cocoyl methyl taurate, sodium lauroyl methyl taurate, sodium cocomo-glyceride sulfate; higher alkyl sulfates, such as sodium lauryl sulfate; higher alkyl-ether sulfates, e.g., of formula CH₃(CH₂)ₘCH₂(OCH₂CH₂)ₙOSO₃X, wherein m is 6-16, e.g., 10, n is 1-6, e.g. 2, 3 or 4, and X is Na, for example sodium laureth-2 sulfate (CH₃(CH₂)₁₀CH₂(OCH₂CH₂)₂OSO₃Na); higher alkyl aryl sulfonates such as sodium dodecyl benzene sulfonate (sodium lauryl benzene sulfonate); higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide), sodium lauryl sarcosinate, or any mixture or combination thereof. As used herein, “higher alkyl” may refer to a C₆-C₃₀ alkyl.

In some embodiments, anionic surfactants utilized in the oral care composition do not include one or more alkyl sulfate salts, such as sodium lauryl sulfate. For example, as further described herein, the oral care composition may be free or substantially free of alkyl sulfate salts, such as sodium lauryl sulfate. As used herein, “free” or “substantially free” of a material may refer to a composition, component, or phase where the material is present in an amount of less than 10.0 weight %, less than 5.0 weight %, less than 3.0 weight %, less than 1.0 weight %, less than 0.1 weight %, less than 0.05 weight %, less than 0.01 weight %, less than 0.005 weight %, or less than 0.0001 weight % based on a total weight of the composition, component, or phase.

The amphoteric and zwitterionic surfactants may be or include, but are not limited to, derivatives of C₈₋₂₀ aliphatic secondary and tertiary amines having an anionic group such as carboxylate, sulfate, sulfonate, phosphate or phosphonate. Illustrative amphoteric and zwitterionic surfactants may include, but are not limited to, sultaines and betaines, such as cocamidopropyl betaine (CAPB), derivatives of aliphatic secondary and tertiary amines in which the aliphatic radical can be a straight or branched chain and wherein one of the aliphatic substituents contains about 8-18 carbon atoms and one contains an anionic water-solubilizing group, such as carboxylate, sulfonate, sulfate, phosphate or phosphonate, and combinations thereof.

In some embodiments, the nonionic surfactants may be or include, but are not limited to, one or more alkyl polyglucosides, such as one or more C6-C24 (e.g., C6-C18) alkyl polyglucosides. The C6-C24 alkyl polyglucosides may include one or more short chain alkyl polyglucosides, one or more long chain alkyl polyglucosides, or any combination thereof. As used herein, the term or expression “short chain alkyl polyglucosides” may refer to alkyl polyglucosides having a chain length of from about 6 to about 10 carbon atoms. As used herein, the term or expression “long chain alkyl polyglucosides” may refer to alkyl polyglucosides having a chain length of from about 11 to about 24 carbon atoms (e.g., from about 11 to about 18 carbon atoms). It should be appreciated that the alkyl polyglucosides may include a hydrophobic fatty alcohol portion and a hydrophilic glucoside portion. In a preferred embodiment, the nonionic surfactants include at least a short chain alkyl polyglucoside, such as a C6-C10 alkyl polyglucoside or a C8-C10 alkyl polyglucoside (CAS 68515-73-1), which is commercially available as APG 8/10. In one embodiment, the oral care composition is free or substantially free of long chain alkyl polyglucosides.

Additional illustrative nonionic surfactants may be or include, but are not limited to, one or more of octoxynol (e.g., Macrogol tetramethylbutylphenyl ether, octylphenoxy polyethoxyethanol, or polyoxyethylene octylphenyl ether), such as octoxynol 1, 3, 5, 8, 9, 10, 12, 13, 16, 30, 40, 70, wherein the number indicates the number of repeating oxyethylene units, or other octoxynols that include different numbers of repeating units of oxyethylene in the side chain, sorbitan esters (e.g., sorbitan monooleate and sorbitan monostearate, etc.) commonly known by their trade names SPAN® 80 and SPAN® 60), polysorbates (e.g., polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of TWEEN® 80, TWEEN® 60, TWEEN® 20), poloxamers (synthetic block polymers of ethylene oxide and propylene oxide, such as those commonly known by their trade names of PLURONIC®; e.g., PLURONIC® F127 or PLURONIC® F108), or poloxamines (synthetic block polymers of ethylene oxide and propylene oxide attached to ethylene diamine, such as those commonly known by their trade names of TETRONIC®; e.g., TETRONIC® 1508 or TETRONIC® 908, etc.), other nonionic surfactants such as BRIJ® (polyoxyethylene alkyl ether having a formula of CH₃—(CH₂)₁₀₋₁₆—(O—C₂H₄)₁₋₂₅—OH), MYRJ® (stearic acid esterified with polyoxyethylene having 40-100 repeating oxyethylene units), long chain fatty alcohols (e.g., oleyl alcohol, stearyl alcohol, myristyl alcohol, docosahexaenoyl alcohol, etc.) with carbon chains having about 12 or more carbon atoms (e.g., such as from about 12 to about 24 carbon atoms), or any mixture or combination thereof. Additional nonionic surfactants may be or include, but are not limited to, polyethylene oxide condensates of alkyl phenols, products derived from the condensation of ethylene oxide with the reaction product of propylene oxide and ethylenediamine, ethylene oxide condensates of aliphatic alcohols, long chain tertiary amine oxides, long chain tertiary phosphine oxides, long chain dialkyl sulfoxides, or combinations thereof. In at least one embodiment, the nonionic surfactants may be broadly defined as compounds produced by the condensation of alkylene oxide groups (hydrophilic in nature) with an organic hydrophobic compound, which may be aliphatic or alkylaromatic in nature. In some embodiments, the oral care composition may be free or substantially free of one or more surfactants capable of or configured to react with one or more cationic or cationically charged ingredients/components of the oral care composition to form insoluble salts. For example, the oral care composition may be free or substantially free of one or more surfactants capable of or configured to react with one or more cationic or cationically charged amino acids, such as arginine. In another embodiment, the oral care composition may be free or substantially free of one or more surfactants capable of or configured to react with one or more cations provided or released by one or more ingredients/components of the oral care composition. For example, the oral care composition may be free or substantially free of one or more surfactants capable of or configured to react with one or more cations provided by one or more salts (e.g., inorganic salts) contained therein. For example, the oral care composition may be free or substantially free of one or more surfactants capable of or configured to react with one or more calcium ions provided by one or more abrasives of the oral care composition, such as calcium carbonate.

As discussed above, in some embodiments, the oral care composition may be free or substantially free of sodium lauryl sulfate or similar anionic surfactants. It should be appreciated that sodium lauryl sulfate and/or anionic surfactants similar thereto may at least partially react with cationically charged species, such as partially cationically charged arginine and/or cations, such as calcium ions from calcium carbonate, to form inorganic salts. It should further be appreciated that the reaction of sodium lauryl sulfate and/or anionic surfactants similar thereto may reduce the availability and/or foaming capacity of the sodium lauryl sulfate and/or anionic surfactants similar thereto; and thus, reduce the foaming of the oral care composition. As such, in at least one exemplary embodiment, the oral care composition may be free or substantially free of sodium lauryl sulfate or similar anionic surfactants.

The amount of any one or more of the surfactants or foaming agents present in the oral care composition or a component (e.g., hydrophilic or hydrophobic phases) thereof may vary widely. In some embodiments, the amount of any one or more of the surfactants or foaming agents present in the oral care composition or the component thereof may be greater than 0.0 weight % or 0.1 weight % and less than or equal to 10.0 weight %, based on a total weight of the oral care composition or the component thereof. For example, the amount of any one or more of the surfactants or foaming agents present in the oral care composition or the component thereof may be from greater than 0 weight %, about 0.1 weight %, about 0.5 weight %, about 1 weight %, about 1.5 weight %, about 2 weight %, or about 2.5 weight % to about 3 weight %, about 3.5 weight %, about 4 weight %, about 4.5 weight %, about 5 weight %, about 8 weight %, or about 10 weight %, based on a total weight of the oral care composition or the component thereof. In another example, the amount of the surfactant present in the oral care composition or the component thereof may be from about 0.5 weight % to about 5 weight %, about 1 weight % to about 4.5 weight %, about 1.5 weight % to about 4 weight %, about 2 weight % to about 3.5 weight %, or about 2.5 weight % to about 3 weight %, based on a total weight of the oral care composition or the component thereof. In some embodiments, each of the one or more of the surfactants or foaming agents may, separately and independently, be present in the oral care composition or the component thereof in an amount of from about 0.01 weight % to about 3 weight %, about 1 weight % to about 2 weight %, about 1.25 weight % to about 1.75 weight %, about 1.5 weight %, about 1 weight %, or about 0.5 weight %, based on a total weight of the oral care composition.

In some embodiments, a weight ratio (e.g., weight %) of one surfactant to another surfactant may vary from about 0.5:1 to about 9:1. For example, the weight ratio of any one surfactant to another surfactant in the oral care composition may be from about 0.5:1, about 0.6:1, about 0.7:1, about 0.8:1, about 0.9:1, about 1:1, about 1.1:1, about 1.2:1, about 1.3:1, about 1.4:1, about 1.5:1, about 1.6:1, about 1.7:1, about 1.8:1, about 1.9:1, or about 2:1 to about 2.1:1, about 2.2:1, about 2.3:1, about 2.4:1, about 2.5:1, about 2.6:1, about 2.7:1, about 2.8:1, about 2.9:1, about 3:1, about 5:1, about 8:1, or about 9:1. In an exemplary embodiment the weight ratio of at least one surfactant to another surfactant may be from about 0.5:1 to about 9:1, about 0.5:1 to about 3:1, about 1.5:1 to about 2.5:1, about 1.8:1 to about 2.2:1, or about 2:1.

In a preferred embodiment, the oral care composition includes a combination of at least two surfactants. In at least one example, the at least two surfactants may include an anionic surfactant and a nonionic surfactant. In another example, the at least two surfactants may include an amino acid surfactant and a nonionic surfactant. The anionic surfactant may be or include, but is not limited to, one or more taurate surfactants, sodium lauroyl methyl taurate or sodium methyl lauroyl taurate, sodium methyl cocoyl taurate or sodium cocoyl methyl taurate, or combinations thereof. The nonionic surfactant may be or include, but is not limited to, one or more alkyl polyglucosides, such as one or more C6-C18 alkyl polyglucosides. In a preferred embodiment, the oral care composition includes one or more taurate surfactants, sodium lauroyl methyl taurate and/or sodium methyl cocoyl taurate, and a C8-C10 alkyl polyglucoside. The weight ratio (e.g., weight %) of the C8-C10 alkyl polyglucoside to the taurate surfactants may be from about 0.25:1 to about 9:1, about 1.5:1 to about 2.5:1, about 1.8:1 to about 2.2:1, or about 2:1.

In some embodiments, the oral care composition of the invention comprises a taurate surfactant represented by formula (1)

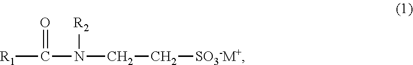

wherein R₁ is a saturated or unsaturated, straight or branched alkyl chain with 6 to 18 C atoms, R₂ is H or methyl, and M⁺ is H, sodium, or potassium. In some embodiments, the taurate surfactant is present in an amount of from 0.4% to 3%, e.g., from 0.4% to 2.5%, from 0.4% to 2%, from 0.4% to 1.5%, from 0.5% to 3%, from 0.8% to 3%, from 1% to 3%, from 1.2% to 2.7%, from 1.5% to 3%, from 2% to 3%, from 1% to 2.8%, from 1% to 2.7%, from 1% to 2.5%, from 1.5% to 2.8%, from 1.5% to 2.5%, from 1.8% to 3%, from 1.8% to 2.8%, from 1.8% to 2.7%, from 1.8% to 2.5%, from 2% to 2.8%, from 2% to 2.7%, or from 2% to 2.5% by weight of the composition.

In some embodiments, the oral care composition of the invention comprises (i) a taurate surfactant represented by formula (1)

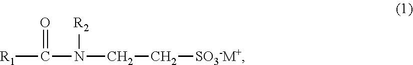

wherein R₁ is a saturated or unsaturated, straight or branched alkyl chain with 6 to 18 C atoms R₂ is H or methyl, and M⁺is H, sodium, or potassium, and (ii) a non-ionic surfactant comprising a C6-C24 alkyl polyglucoside. In some embodiments, the taurate surfactant is present in an amount of from 0.4% to 2%, e.g., from 0.4% to 1.5%, from 0.4 to 1.2%, from 0.5% to 1%, or about 1% by weight of the composition, and the nonionic surfactant is present in an amount of 0.7% to 2.5%, 0.8% to 2.5%, 1% to 2.5%, 1.3% to 2.5%, 1.5% to 2.5%, 0.7% to 2%, 0.8% to 2%, 1% to 2%, 1.3% to 2%, 1.5% to 2%, 0.8% to 1.7%, 0.8% to 1.5%, 1% to 1.5%. 1.3% to 1.8%, 1.3% to 1.7%, or about 1.5% by weight of the composition. In some embodiments, the taurate surfactant is present in an amount of from 0.4% to 1.5% by weight of the composition, and the nonionic surfactant is present in an amount of 0.7% to 2% by weight of the composition. In some embodiments, the taurate surfactant is present in an amount of from 0.8% to 1.2% by weight of the composition, and the nonionic surfactant is present in an amount of 1.3% to 1.8% by weight of the composition. In some embodiments, the taurate surfactant is present in an amount of from 0.5% to 1% by weight of the composition, and the nonionic surfactant is present in an amount of 1% to 1.5% by weight of the composition. In a particular embodiment, the taurate surfactant is present in an amount of about 1% by weight of the composition, and the nonionic surfactant is present in an amount of aboutl.5% by weight of the composition In some embodiments, a weight ratio of the nonionic surfactant to the taurate surfactant is from 0.25:1 to 9:1, e.g., from 1:1 to 5:1, from 1:1 to 2.5:1, from 1.3 to 2.5:1, from 1.3:1 to 2.2:1, from 1.3:1 to 2:1, from 1.4:1 to 1.8:1, from about 1.8:1 to about 2.2:1, from 1.4:1 to 2.1:1, from 1.4:1 to 1.6:1. or from 1.5:1 to 2:1.

### Fluoride Ion Source

In some embodiments, the oral care composition may be free or substantially free of fluoride (e.g., soluble fluoride salts). In another embodiment, the oral care composition may include fluoride, such as one or more fluoride ion sources (e.g., soluble fluoride salts). A wide variety of fluoride ion-yielding materials may be employed as sources of soluble fluoride. Examples of suitable fluoride ion-yielding materials may be found in U.S. Pat. No. 3,535,421 to Briner et al., U.S. Pat. No. 4,885,155 to Parran, Jr. et al., and U.S. Pat. No. 3,678,154 to Widder et al., the disclosures of which are incorporated herein by reference. Illustrative fluoride ion sources include, but are not limited to, fluoride, stannous fluoride, sodium fluoride, potassium fluoride, sodium monofluorophosphate, fluorosilicate salts, such as sodium fluorosilicate and ammonium fluorosilicate, amine fluoride, ammonium fluoride, and combinations thereof. In a typical embodiment, the fluoride ion source includes sodium monofluorophosphate. The amount of the fluoride ion source in the oral care composition may be greater than 0 weight % and less than 0.8 wt %, less than 0.7 wt %, less than 0.6 wt %, less than 0.5 wt %, or less than 0.4 wt %. The fluoride ion sources may be present in an amount sufficient to provide a total of about 100 to about 20,000 ppm, about 200 to about 5,000 ppm, or about 500 to about 2,500 ppm fluoride ions.

### Orally Acceptable Vehicle or Carrier

The oral care composition may form at least a portion of or be used in one or more oral care products. The oral care composition may include or be combined with an orally acceptable vehicle. For example, the oral care composition may include or be combined with an orally acceptable vehicle to form the oral care product. The orally acceptable vehicle may include any known ingredients or additives. The orally acceptable vehicle may include various dentifrice ingredients to adjust the rheology and feel of the oral care composition.
In at least one embodiment, the orally acceptable vehicle may include one or more humectants. Illustrative humectants may be or include, but are not limited to, glycerin, propylene glycol, polyethylene glycol, sorbitol, xylitol, or any mixture or combination thereof. In a preferred embodiment, the orally acceptable vehicle may be or include, but is not limited to, sorbitol. The one or more humectants may be present in the oral care composition in an amount of from about 5 weight % to about 35 weight %, based on a total weight of the oral care composition.

In at least one embodiment, the orally acceptable vehicle may include an orally acceptable solvent. Illustrative solvents may include, but are not limited to, one or more of ethanol, phenoxyethanol, isopropanol, water, cyclohexane, methyl glycol acetate, benzyl alcohol, or any mixture or combination thereof. In a preferred embodiment, the orally acceptable solvent includes benzyl alcohol.

The orally acceptable vehicle may be present in an amount of from 5 weight % to about 60 weight %, based on a total weight of the oral care composition. For example, the orally acceptable vehicle may be present in an amount of from about 5 weight %, about 10 weight %, about 15 weight %, or about 20 weight % to about 25 weight %, about 30 weight %, about 35 weight %, about 40 weight %, about 45 weight %, about 50 weight %, about 55 weight %, or about 60 weight %. In another example, the orally acceptable vehicle may be present in an amount of from about 5 weight % to about 60 weight %, about 10 weight % to about 55 weight %, about 15 weight % to about 50 weight %, about 20 weight % to about 25 weight %, about 20 weight % to about 40 weight %, about 20 weight % to about 35 weight %, about 20 weight % to about 30 weight %, or about 20 weight % to about 25 weight %. In an exemplary embodiment, the orally acceptable vehicle may be present in an amount of about 20 weight % to about 30 weight %, preferably about 20 weight % to about 25 weight %, and more preferably about 22 weight % to about 25 weight %. In a preferred embodiment, the orally acceptable vehicle may be present in an amount of about 22 weight % to about 25 weight % or about 23 weight %.

Thickening System and/or Viscosity Control Agents

The oral care product or the oral care composition thereof may include a thickening system having one or more thickeners. The one or more thickeners may be any orally acceptable thickener or thickening agent configured to control the viscosity of the oral care product or the oral care composition thereof. Illustrative thickeners may be or include, but are not limited to, colloidal silica, fumed silica, a cross-linked polyvinylpyrrolidone (PVP) polymer, cross-linked polyvinylpyrrolidone (PVP), or mixtures or combinations thereof. In at least one embodiment, the thickening system includes a cross-linked polyvinylpyrrolidone (PVP) polymer. The thickening system may also include POLYPLASDONE® XL 10F, which is commercially available from Ashland Inc. of Covington, Ky. Illustrative thickeners may also be or include, but are not limited to, carbomers (e.g., carboxyvinyl polymers), carrageenans (e.g., Irish moss, carrageenan, iota-carrageenan, etc.), high molecular weight polyethylene glycols (e.g., CARBOWAX®, which is commercially available from The Dow Chemical Company of Midland, Mich.), cellulosic polymers, hydroxyethylcellulose, carboxymethylcellulose, and salts thereof (e.g., CMC sodium), natural gums (e.g., karaya, xanthan, gum arabic, and tragacanth), colloidal magnesium aluminum silicate, or mixtures or combinations thereof.

In a more typical embodiment, the thickening system may include an organic polymer, which may also be configured as an adhesion enhancing agent. Illustrative organic polymers may be or include, but are not limited to, hydrophilic polymers, such as carbomers, such as carboxymethylene polymers, such as acrylic acid polymers, and acrylic acid copolymers. Carboxypolymethylene is a slightly acidic vinyl polymer with active carboxyl groups. In a typical embodiment, the thickening system includes a carboxypolymethylene, such as CARBOPOL® 974 and/or 980, which are commercially available from Noveon, Inc. of Cleveland, Ohio.

In at least one embodiment, the thickening system may include a single thickener. For example, the thickening system may include the cross-linked polyvinylpyrrolidone (PVP) polymer or an organic polymer (e.g.,)CARBOPOL®. In another embodiment, the thickening system may include a plurality of thickeners. For example, the thickening system may include the cross-linked PVP polymer and the organic polymer.

The amount or concentration of the thickening system and/or the thickeners thereof present in the oral care composition may vary widely. The amount of the thickening system and/or the thickeners thereof present in the oral care composition may from about 1.0 weight % to about 3.0 weight % based on the total weight of the oral care composition. For example, the amount of the thickening system and/or the thickeners thereof present in the oral care composition may be from about 1 weight %, about 1.1 weight %, about 1.2 weight %, about 1.3 weight %, about 1.4 weight %, about 1.5 weight %, about 1.6 weight %, about 1.7 weight %, about 1.8 weight %, about 1.9 weight %, about 2.0 weight %, or about 2.1 weight % to about 2.2 weight %, about 2.3 weight %, about 2.4 weight %, about 2.5 weight %, about 2.6 weight %, about 2.7 weight %, about 2.8 weight %, about 2.9 weight %, or about 3.0 weight %. In another example, the amount of the thickening system and/or the thickeners thereof present in the oral care composition may from about 1.2 weight % to about 3.0 weight %, about 1.3 weight % to about 2.9 weight %, about 1.4 weight % to about 2.8 weight %, about 1.5 weight % to about 2.7 weight %, about 1.6 weight % to about 2.6 weight %, about 1.7 weight % to about 2.5 weight %, about 1.8 weight % to about 2.4 weight %, about 1.9 weight % to about 2.3 weight %, or about 2.0 weight % to about 2.2 weight %. In a typical embodiment, the amount of the thickening system and/or the thickeners thereof present in the oral care composition may be from about 1.0 weight % to about 2.0 weight %, more typically about 1.2 weight % to about 1.8 weight %, and more typically about 1.5 weight %.

### pH Modifying Agents

The oral care product or the oral care composition or a component thereof may include one or more pH modifying agents. For example, the oral care composition may include one or more acidifying agents and/or one or more basifying agents configured to reduce and/or increase the pH thereof, respectively. Illustrative acidifying agents and/or one or more basifying agents may be or include, but are not limited to, an alkali metal hydroxide, such as sodium hydroxide and/or potassium hydroxide, citric acid, hydrochloric acid, or combinations thereof.

The oral care composition or a component thereof may also include one or more buffering agents configured to control or modulate the pH within a predetermined or desired range. Illustrative buffering agents may include, but are not limited to, sodium bicarbonate, sodium phosphate, sodium carbonate, sodium acid pyrophosphate, sodium citrate, and mixtures thereof. Sodium phosphate may include monosodium phosphate (NaH₂PO₄), di sodium phosphate (Na₂HPO₄), trisodium phosphate (Na₃PO₄), and mixtures thereof. In a typical embodiment, the buffering agent may be anhydrous sodium phosphate dibasic or disodium phosphate and/or sodium phosphate monobasic. In another embodiment, the buffering agent includes anhydrous sodium phosphate dibasic or &sodium phosphate, and phosphoric acid syrupy phosphoric acid; 85%-Food (parade).

In at least one embodiment, the acidifying, buffering, and/or buffering agents may be included in the oral care composition or a component thereof to provide a generally neutral pH or an orally acceptable pH range. In another embodiment, the acidifying, buffering, and/or buffering agents may be included in the oral care composition or a component thereof (e.g., hydrophobic and/or hydrophilic phases) with a pH between 2 to 10, 2 to 8, 3 to 9, 4 to 8, 6 to 10, or 7 to 9. Any additional orally acceptable pH modifying agent may be used, including without limitation carboxylic, phosphoric, and sulfonic acids, acid salts (e.g., monosodium citrate, disodium citrate, monosodium malate, etc.), alkali metal hydroxides, such as sodium hydroxide, carbonates, such as sodium carbonate, bicarbonates, sesquicarbonates, borates, silicates, phosphates (e.g., monosodium phosphate, trisodium phosphate, pyrophosphate salts, etc.), imidazole and mixtures thereof. The one or more pH modifying agents may be optionally present in an amount effective to maintain the oral care composition or a component thereof in an orally acceptable pH range.

### Flavoring Agents

The oral care product and/or the oral care composition thereof may also include one or more flavoring agents. Illustrative flavoring agents may include, but are not limited to, essential oils and various flavoring aldehydes, esters, alcohols. The flavoring agents may also include, but are not limited to, sweeteners, sucralose, dextrose, polydextrose, sucrose, maltose, dextrin, dried invert sugar, mannose, xylose, ribose, fructose, levulose, galactose, corn syrup (including high fructose corn syrup and corn syrup solids), partially hydrolyzed starch, hydrogenated starch hydrolysate, sorbitol, mannitol, xylitol, maltitol, isomalt, aspartame, neotame, saccharin and salts thereof (e.g., sodium saccharin), dipeptide-based intense sweeteners, cyclamates, dihydrochalcones and mixtures thereof. Examples of the essential oils include oils of spearmint, peppermint, wintergreen, sassafras, clove, sage, eucalyptus, marjoram, cinnamon, lemon, lime, grapefruit, and orange. In another example, the flavoring agents may include menthol, carvone, and anethole. In a typical embodiment, the flavoring agent includes peppermint and spearmint. In a more typical embodiment, the flavoring agent includes a Firmenich Newman Flavor. The amount of the flavoring agent in the oral care product and/or the oral care composition thereof may be less than 1.0 wt %, less than 0.9 wt %, less than 0.8 wt %, or less than 0.7 wt %. For example, the amount of the flavoring agent in the oral care product and/or the oral care composition thereof may be about 0.0 wt % to about 1.0 wt %, about 0.5 wt % to about 0.9 wt %, about 0.7 wt % to about 0.8 wt %. In a typical embodiment, the amount of the flavoring agent in the oral care product and/or the oral care composition thereof is about 0.55 wt % to about 0.70 wt %.

### Additional Ingredients

It should be appreciated to one having ordinary skill in the art, that the oral care products and/or the oral care composition thereof may include other additional ingredients/components. For example, the oral care products and/or the oral care composition thereof may include any one or more of anti-caries agents, desensitizing agents, viscosity modifiers, diluents, pH modifying agents, humectants, mouth feel agents, sweetening agents, flavor agents, colorants, preservatives, or combinations and mixtures thereof. It should further be appreciated by one having ordinary skill in the art that while general attributes of each of the above categories of materials may differ, there may be some common attributes and any given material may serve multiple purposes within two or more of such categories of materials.

In at least one embodiment, the additional ingredients/components may include one or more active materials configured to prevent and/or treat one or more conditions and/or disorders of the oral cavity. For example, the one or more active materials may be configured to prevent and/or treat one or more conditions and/or disorders of hard and/or soft tissue of the oral cavity, such as dentinal hypersensitivity. The active materials may also be configured to prevent and/or treat one or more physiological disorders and/or conditions, and/or provide a cosmetic benefit to the oral cavity.

In at least one embodiment, the oral care products or the oral care composition thereof may include an anticalculus agent. Illustrative anticalculus agents may include, but are not limited to, phosphates and polyphosphates (e.g., pyrophosphates), polyaminopropanesulfonic acid (AMPS), hexametaphosphate salts, zinc citrate trihydrate, polypeptides, polyolefin sulfonates, polyolefin phosphates, diphosphonates. In a typical embodiment, the anticalculus agent includes tetrasodium pyrophosphate (TSPP), sodium tripolyphosphate (STPP), or a combination thereof.

The oral care products or the oral care composition thereof may include an antioxidant. Any orally acceptable antioxidant may be used, including, but not limited to, butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, or combinations and mixtures thereof.

The oral care composition may include zinc. The zinc of the oral care composition may be or include a zinc ion and/or one or more zinc salts. For example, the zinc salts may at least partially dissociate in an aqueous solution to produce zinc ions. Illustrative zinc salts may include, but are not limited to, zinc lactate, zinc oxide, zinc chloride, zinc phosphate, zinc citrate, zinc acetate, zinc borate, zinc butyrate, zinc carbonate, zinc formate, zinc gluconate, zinc glycerate, zinc glycolate, zinc picolinate, zinc proprionate, zinc salicylate, zinc silicate, zinc stearate, zinc tartrate, zinc undecylenate, and mixtures thereof. In a preferred embodiment, the zinc salt is selected from zinc oxide, zinc citrate, and a combination thereof. In some embodiments, the zinc ion source is present in an amount of from 0.01% to 5%, e.g., 0.1% to 4%, or 1% to 3%, by weight of the composition.

In some embodiments, the oral care composition comprises zinc oxide. Zinc oxide may be present in an amount of 0.5% to 2%, e.g., 0.5% to 1.5%, about 1% or about 1.2% by weight of the composition. In some embodiments, the oral care composition comprises zinc citrate. Zinc citrate may be present in an amount of 0.1%-1%, 0.25-0.75%, about 0.5% by weight of the composition by weight of the composition. In some embodiments, the composition comprises zinc oxide and zinc citrate. The compositions may comprise zinc oxide in an amount of 0.5% to 2%, e.g., 0.5% to 1.5%, about 1% or about 1.2% by weight of the composition and zinc citrate in an amount of 0.1%-1%, 0.25-0.75%, about 0.5% by weight of the composition. In some embodiments, the compositions comprise zinc oxide in an amount of about 1% by weight of the composition and zinc citrate in an amount of about 0.5% by weight of the composition.

In some embodiments, the dentifrice composition comprises zinc phosphate. In some embodiments, the composition may comprise zinc phosphate in an amount of 0.5% to 2%, e.g., 0.5% to 1.5%, about 1% or about 1.2% by weight of the composition.

The oral care composition may include one or more pigments, such as whitening pigments. In some embodiments, the whitening pigments include particles ranging in size from about 0.1 μm to about 10 μm with a refractive index greater than about 1.2, Suitable whitening agents include, without limitation, titanium dioxide particles, zinc oxide particles, aluminum oxide particles, tin oxide particles, calcium oxide particles, magnesium oxide particles, barium oxide particles, silica particles, zirconium silicate particles, mica particles, talc particles, tetracalcium phosphate particles, amorphous calcium phosphate particles, alpha-tricalcium phosphate particles, beta-tricalcium phosphate particles, hydroxyapatite particles, calcium carbonate particles, zinc phosphate particles, silicon dioxide particles, zirconium silicate particles, or mixtures and combinations thereof. The whitening pigment, such as titanium dioxide particles, may be present in an amount that is sufficient to whiten the teeth.

### Methods

The present disclosure provides methods comprising applying an effective amount of an oral care product and/or the oral care composition thereof as disclosed herein to the oral cavity, e.g., by brushing, to a human or animal subject in need thereof, to (i) reduce or inhibit formation of dental caries, (ii) reduce, repair or inhibit pre-carious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the oral cavity, (vii) reduce levels of acid producing bacteria, (viii) reduce or inhibit microbial biofilm formation in the oral cavity, (ix) reduce or inhibit plaque formation in the oral cavity, (x) promote systemic health, or (xi) clean teeth and oral cavity. In a preferred embodiment, the present disclosure provides methods for treating dentinal hypersensitivity and/or cleaning teeth in a human or animal subject with an oral care product and/or the oral care composition thereof as disclosed herein. As used herein “animal subject” may include higher order non-human mammals such as canines, felines, and horses. The method may include contacting the oral care product and/or the oral care composition thereof with water. The method may also include contacting the surface of the teeth with the oral care product and/or the oral care composition thereof. Contacting the surface of the teeth with the oral care product and/or the oral care composition thereof may include disposing the oral care composition (e.g., toothpaste) on a toothbrush and brushing the teeth with the toothbrush.

The oral care product and/or the oral care composition thereof may be applied and/or contacted with the surfaces of the teeth at predetermined intervals. For example, a daily basis, at least once a day, twice a day, or more, for multiple days, or alternatively every other day. In another example, the oral care product and/or the whitening composition thereof may be applied and/or contacted with the surfaces of the teeth at least once a day, at least once every two days, at least once every three days, at least once every five days, at least once a week, at least once every two weeks, or at least once a month. The oral care product and/or the oral care composition thereof may be utilized for up to 2 weeks, up to 3 weeks, up to 4 weeks, up to 6 weeks, up to 8 weeks, or greater.

The present disclosure may also provide methods for preparing oral care compositions having increased foam generation as compared to conventional oral care compositions and/or methods for increasing foam generation in oral care compositions relative to conventional oral care compositions. The method may include combining or contacting an orally acceptable vehicle with at least two surfactants, including an anionic surfactant and a nonionic surfactant, where the anionic surfactant includes one or more amino acid surfactant, such as one or more taurate surfactant, and where the nonionic surfactant includes an alkyl polyglucosides, such as a C6-C24 alkyl polyglucoside. The method may further include combining the orally acceptable vehicle, the anionic surfactant, and the nonionic surfactant with one or more amino acids and/or one or more abrasives. For example, the method may include combining the orally acceptable vehicle, the anionic surfactant, and the nonionic surfactant with arginine or a source of arginine and calcium carbonate.

All ingredients for use in the compositions described herein should be orally acceptable. As used herein, “orally acceptable” may refer any ingredient that is present in a composition as described in an amount and form which does not render the composition unsafe for use in the oral cavity.

### EXAMPLES

The examples and other embodiments described herein are exemplary and not intended to be limiting in describing the full scope of compositions and methods of this disclosure. Equivalent changes, modifications and variations of specific embodiments, materials, compositions and methods may be made within the scope of the present disclosure, with substantially similar results.

### Example 1

The foam volume generated by oral care compositions containing arginine bicarbonate and precipitated calcium carbonate was evaluated with variation of surfactants to determine the impact of different surfactants on foam generation. A negative control toothpaste compositions (C1) and seven test toothpaste compositions 1-7 were prepared by combining the ingredients/components of a base toothpaste composition indicated in Table 1, with respective surfactant(s) indicated in Table 2. The components were mixed for about 10 minutes under mechanical stirring to prepare each of the toothpaste compositions C1 and 1-7. Notably, the negative control toothpaste composition (C1) contained sodium lauryl sulfate (SLS) as the surfactant and Compositions 1-7 contained various amounts of Sodium Lauroyl Methyl Taurate (SLMT) and/or Alkyl Polyglucoside-08/10 (APG). Colgate Max Fresh toothpaste (C2) was used as positive control for foam generation. The positive control toothpaste composition C2 contained a combination of SLS and cocoamidopropyl betaine (CAP Betaine).

TABLE 1
Base Toothpaste Composition

| | Concentration |
|---|---|
| INGREDIENT/COMPONENT | (Weight %) |
| Sorbitol - non-crystal - 70% solution | 23.0% |
| Demineralized Water | 17.3% -surfactants % |
| Benzyl Alcohol | 0.3% |
| Arginine Bicarbonate solution 40.8% | 19.6% |
| Sodium Monofluorophosphate | 1.1% |
| Tetrasodium pyrophosphate | 0.5% |
| Sodium Bicarbonate | 0.5% |
| Precipitated calcium carbonate - | 10.0% |
| Medium Absorption | |
| Precipitated calcium carbonate - | 25.0% |
| High Absorption | |
| Titanium Dioxide (TiO₂) | 0.5% |
| Excipients | Balance |

TABLE 2
Surfactant(s) Added to Base Toothpaste Composition
to Prepare Control Toothpaste Compositions C1
and C2 and Exemplary Toothpaste Compositions 1-7

| SURFACTANTS | (C1) | (C2) | (1) | (2) | (3) | (4) | (5) | (6) | (7) |
|---|---|---|---|---|---|---|---|---|---|
| 30% Sodium Lauryl | 4.9 | 6.6 | — | — | — | — | — | — | — |
| Sulfate (SLS) (g) | | | | | | | | | |
| 30% Cocoamidopropyl | — | 1.7 | — | — | — | — | — | — | — |
| Betaine (g) | | | | | | | | | |
| Sodium Lauroyl | — | — | 1.5 | — | 2.0 | 2.5 | — | 0.5 | 1.0 |
| Methyl Taurate | | | | | | | | | |
| (SLMT) (g) | | | | | | | | | |
| 30% Sodium Cocoyl | — | — | — | — | — | — | — | — | — |
| Methyl Taurate | | | | | | | | | |
| (SCMT) (g) | | | | | | | | | |
| 64% Alkyl | — | — | — | 2.4 | — | — | 3.9 | 1.6 | 2.4 |
| Polyglucoside-08/10 | | | | | | | | | |
| (APG) (g) | | | | | | | | | |
| Total Concentration | 1.5 | 2.5 | 1.5 | 1.5 | 2.0 | 2.5 | 2.5 | 1.5 | 2.5 |
| of Surfactants | | | | | | | | | |
| (weight %) | | | | | | | | | |

To evaluate the efficacy for producing foam, a Kruss Dynamic Foam Analyzer (DFA100), commercially available from Kruss GmbH of Hamburg, Germany, was utilized. The Kruss Dynamic Foam Analyzer was adjusted according to the parameters/testing conditions indicated in Table 3.

TABLE 3
Parameters of Krüss Dynamic Foam Analyzer

| PARAMETER | TESTING CONDITION | |
|---|---|---|
| Sample Concentration | 30% | |
| Stirring Speed | 5,000 | RPM |
| Oscillation Intervals | 6 | sec |
| Foam Time | 60 | sec |
| Delay Time | 450 | sec |

The results of the foam generation are summarized in Table 4.

TABLE 4
Summary of Foam Generation in Control Toothpaste Compositions
C1 and C2 and Exemplary Toothpaste Compositions 1-7

| | | Maximum Foam Volume |
|---|---|---|
| Composition | Active Foaming Agent | (mL) |
| C1 | 1.5% SLS | 77.6 |
| C2 | 2.0% SLS + 0.5% CAP Betaine | 89.0 |
| 1 | 1.5% SLMT | 84.3 |
| 2 | 1.5% APG 08/10 | 50.2 |
| 3 | 2.0% SLMT | 90.7 |
| 4 | 2.5% SLMT | 96.8 |
| 5 | 2.5% APG 08/10 | 58.2 |
| 6 | 0.5% SLMT + 1% APG 08/10 | 82.7 |
| 7 | 1.0% SLMT + 1.5% APG 08/10 | 100.0 |

As shown in Table 4, Composition 1 (1.5% SLMT) generated more foam than composition C1 (1.5% SLS). Increasing the amount of SLMT to 2.0% and 2.5% (Compositions 3 and 4, respectively) improved foaming production significantly. These results show that the taurate surfactant is superior in terms of foam volume when compared to SLS in the PCC based toothpaste containing arginine, although both taurate surfactant and SLS are anionic surfactants. In order to explore why taurate surfactant helps in boosting the foam in comparison to SLS, CaCl₂ (source of soluble calcium ions) and arginine solution was reacted with taurate surfactant solution. A clear solution was obtained from the reaction.

{CaCl₂+L-Arginine}+Taurate surfactant→Clear solution

On the other hand, when CaCl₂ and arginine solution were added to SLS solution, a white precipitate of (Ca(H₂Arg)₂)(LS)₄ products was yielded. These results show that taurate surfactant is a much better alternative to sodium lauryl sulfate in the presence of arginine and calcium carbonate in order to boost the foam volume.

Table 4 further shows that composition 6 (0.5% SLMT+1% APG 08/10) exhibited relatively greater foam production (1.5% SLS) (82.7 mL) than composition C1(77.6 mL). Increasing the amounts of the SLMT and APG 08/10, as in test toothpaste composition 7 (1.0% SLMT +1.5% APG 08/10) improved foam production (100 mL) significantly. Notably, the combination of the Taurate foaming agent and APG, at an overall concentration of 2.5%, as in Composition 7 exhibited more foaming as compared to Compositions 4 (2.5% SLMT). This result is surprising, because APG 08/10 alone exhibits poor foaming activity. Table 4 shows that utilizing APG 08/10 alone, as in composition 2 (1.5% APG 08/10) and 5 (2.5% APG 08/10), did not result in good foam production (50.2 mL and 58.2 mL, respectively). These results show that the combination of a taurate surfactant and an alkyl polyglucoside surfactant exhibits synergistic or more than additive foaming as compared to each of the surfactants alone.

The present disclosure has been described with reference to exemplary embodiments. Although a limited number of embodiments have been shown and described, it will be appreciated by those skilled in the art that changes may be made in these embodiments without departing from the principles and spirit of the preceding detailed description. It is intended that the present disclosure be construed as including all such modifications and alterations insofar as they come within the scope of the appended claims or the equivalents thereof.

1. An oral care composition comprising:
an orally acceptable vehicle;
a taurate surfactant represented by formula (1) 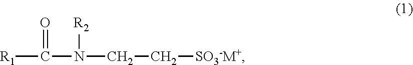
wherein R₁ is a saturated or unsaturated, straight or branched alkyl chain with 6 to 18 C atoms R₂ is H or methyl, and M⁺ is H, sodium, or potassium;
a basic amino acid, in free or salt form; and
an abrasive.

2. The oral care composition of claim 1, wherein the R₁ is a saturated or unsaturated, straight or branched alkyl chain with 8 to 14 C atoms, optionally wherein the taurate surfactant comprises one or more of potassium cocoyl taurate, potassium methyl cocoyl taurate, sodium caproyl methyl taurate, sodium cocoyl taurate, sodium lauroyl taurate, sodium methyl cocoyl taurate, sodium methyl lauroyl taurate, sodium methyl myristoyl taurate, sodium methyl oleoyl taurate, sodium methyl palmitoyl taurate, sodium methyl stearoyl taurate, or combinations and mixtures thereof, and further optionally wherein the taurate surfactant comprises sodium lauroyl methyl taurate or sodium methyl lauroyl taurate, sodium methyl cocoyl taurate or sodium cocoyl methyl taurate, or combinations thereof.

3. The oral care composition of claim 1, wherein the taurate surfactant is present in an amount of from 0.4% to 3% by weight of the composition.

4. The oral care composition of claim 1, further comprising a nonionic surfactant, optionally wherein the nonionic surfactant comprises a C6-C24 alkyl polyglucoside.

5. The oral care composition of claim 4, wherein the C6-C24 alkyl polyglucoside comprises a short chain alkyl polyglucoside, optionally wherein the short chain alkyl polyglucoside comprises a C6-C10 alkyl polyglucoside, and further optionally wherein the short chain alkyl polyglucoside comprises a C8-C10 alkyl polyglucoside.

6. (canceled)

7. The oral care composition of claim 4, wherein the nonionic surfactant is present in an amount of from 0.7% to 2.5% by weight of the composition.

8. The oral care composition of claim 4, wherein a weight ratio of the nonionic surfactant to the taurate surfactant is from 0,25:1 to 9:1, optionally from 1.3:1 to about 2.2:1.

9. The oral care composition of claim 1, wherein the basic amino acid comprises one or more of arginine, lysine, citrulline, ornithine, creatine, histidine, diaminobutanoic acid, diaminoproprionic acid, salts thereof, or combinations thereof.

10. The oral care composition of claim 9, wherein the basic amino acid comprises arginine.

11. The oral care composition of claim 10, wherein the arginine is arginine bicarbonate.

12. The oral care composition of claim 1, the basic amino acid is present in an amount of from 1% to 15% by weight of the composition, being calculated as free base form.

13. The oral care composition of claim 1, wherein the abrasive comprises a calcium-containing abrasive.

14. The oral care composition of claim 13, wherein the abrasive comprises calcium carbonate, optionally wherein the calcium carbonate comprises precipitated calcium carbonate.

15. The oral care composition of claim 1, wherein the abrasive is present in an amount of from 15% to 70% by weight of the composition.

16. The oral care composition of claim 1, wherein the oral care composition is substantially free of alkyl sulfate salts.

17. The oral care composition of claim 1, wherein the oral care composition is a toothpaste.

18. The oral care composition of claim 1, wherein the oral care composition comprises:
a. an orally acceptable vehicle;
b. sodium lauroyl methyl taurate in an amount from 0.25%-1.25% by wt. of the composition;
c. C8-C10 alkyl polyglucoside in an amount from 0.75% -1.75% by wt. of the composition;
d. arginine; and
e. calcium carbonate

19. The oral care composition of claim 1, wherein the oral care composition comprises:
a. an orally acceptable vehicle;
b. sodium lauroyl methyl taurate in an amount of about 0.5% by wt. of the composition;
c. C8-C10 alkyl polyglucoside in an amount from about 1.0% by wt. of the composition
d. arginine; and
e. calcium carbonate

20. The oral care composition of claim 1, wherein the oral care composition comprises:
a. An orally acceptable vehicle;
b. sodium lauroyl methyl taurate in an amount of about 1.0% by wt. of the composition;
c. C8-C10 alkyl polyglucoside in an amount from about 1.5% by wt. of the composition
d. arginine; and
e. calcium carbonate

21. A method for treating dentinal hypersensitivity in a human, the method comprising contacting the oral care composition of claim 1 with surfaces of teeth of the human.
